(19) United States
(12) Patent Application Publication
(10) Pub. No.: US 2011/0034973 A1
(43) Pub. Date: Feb. 10, 2011

(54) Medical Laser Apparatus with Output Beam Homogenizer

(75) Inventor: Sean Xiaolu Wang, Wilmington, DE (US)

Correspondence Address: BWT PROPERTY, INC., 19 SHEA WAY, SUITE 301, NEWARK, DE, 19713, US

(73) Assignee: BWT PROPERTY, INC., Newark, DE (US)

(21) Appl. No.: 12/850,658

(22) Filed: Aug. 5, 2010

Related U.S. Application Data
(60) Provisional application No. 61/231,695, filed on Aug. 6, 2009.

(51) Int. Cl. A61N 5/06

(52) U.S. Cl. 607/89

(57) ABSTRACT

A medical laser apparatus with a beam homogenizer for producing a uniform output beam profile. The beam homogenizer comprises a light pipe which expands the laser beam and mixes the laser light through total internal reflection as well as an optical diffuser to provide further control of the intensity distribution of the laser beam.

### REFERENCE TO RELATED APPLICATION

This application claims an invention which was disclosed in Provisional Patent Application No. 61/231,695, filed Aug. 6, 2009, entitled “MEDICAL LASER APPARATUS WITH OUTPUT BEAM HOMOGENIZER”. The benefit under 35 USC §119(e) of the above mentioned U.S. Provisional Applications is hereby claimed, and the aforementioned application is hereby incorporated herein by reference.

### FIELD OF THE INVENTION

This invention generally relates to a medical laser apparatus, and more specifically to a medical laser apparatus with output beam homogenizer.

### BACKGROUND

Due to modal interference, the output beam of a multimode laser light source (such as a high power multimode laser diode) is generally non-uniform in intensity distribution. This non-uniformity can be even worse when the laser light is delivered through multi-mode optical waveguides, such as multi-mode optical fibers. Even for a single mode laser, the output beam profile follows a Gaussian type intensity distribution, which is far from uniform. Yet in many medical applications such as laser therapy, laser biostimulation, and photo dynamic therapy, it is highly desirable that the laser light source has a uniform output beam profile.

### SUMMARY OF THE INVENTION

It is thus the overall goal of the present invention to solve the above-mentioned problem and provide a medical laser apparatus with a beam homogenizer for producing a uniform output beam profile. The beam homogenizer comprises a light pipe which expands the laser beam and mixes the laser light through total internal reflection as well as an optical diffuser to provide further control of the intensity distribution of the laser beam. The beam homogenization scheme is especially suitable for ultra high power lasers, for which conventional laser beam shaping techniques can not be applied.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the medical laser apparatus with output beam homogenizer.

Skilled artisans will appreciate that elements in the FIGURE are illustrated for simplicity and clarity and have not necessarily been drawn to scale. For example, the dimensions of some of the elements in the FIGURE may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a medical laser apparatus with output beam homogenizer. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

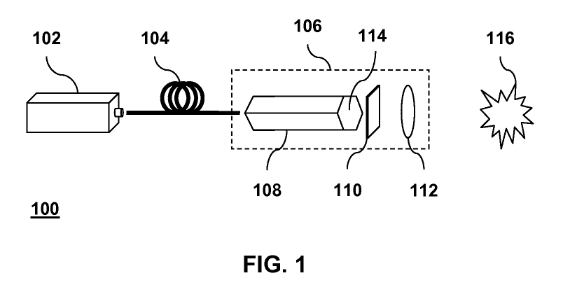

One exemplary embodiment of the present invention is shown in FIG. 1. The medical laser apparatus 100 comprises a high power multimode diode laser unit 102, which produce a laser beam with a power level in the range of a few watts to several tens of watts. The laser light is coupled into a multimode optical fiber 104 and delivered to an output beam homogenization unit 106. The beam homogenization unit 106 comprises a hexagonal shaped fused silica light pipe 108 and an optical diffuser 110. The hexagonal light pipe 108 functions as the first stage beam homogenizer, which expands the laser beam and mixes the laser light through total internal reflection occurring at its six side surfaces. As a result, the output laser beam from the light pipe 108 exhibits lower power density and more uniform intensity distribution. The optical diffuser 110, consisting of a large number of microstructures (such as micro-lenses) on its surface, functions as the second stage beam homogenizer to further homogenize the laser beam. Some possible choices for the optical diffuser 110 include holographic diffusers and those micro-lens arrays created by laser lithography. The incident light intensity on the optical diffuser 110 is greatly reduced by the light pipe 108, which helps to avoid any thermal damage to the optical diffuser 110. This feature allows the present beam homogenization scheme to handle very high laser powers. The beam homogenization unit 106 further comprises an optical lens 112, which is used to project an image of the output surface 114 of the light pipe 108 onto the target surface 116 to form a uniform laser illumination. The spot size and intensity of the laser beam on the target surface 116 can be controlled by selecting proper focal length for the optical lens 112 and/or by adjusting its relative position to the output surface 114 of the light pipe 108. In a slight variation of the present embodiment, the hexagonal light pipe 108 may be replaced with other types of multimode light guides, such as liquid light guides to achieve similar beam expansion and homogenization effects.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and FIGURE are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A medical laser apparatus with a uniform output beam profile, the medical laser apparatus comprising:
a laser light source for producing a laser beam;
a light pipe positioned in a path of said laser beam for mixing light of said laser beam through total internal reflection and expanding said laser beam to produce an expanded laser beam; and
an optical diffuser for homogenizing said expanded laser beam to produce an output beam with uniform intensity distribution.

2. The medical laser apparatus of claim 1, further comprising an optical lens for projecting said output beam onto a target surface.

3. The medical laser apparatus of claim 1, wherein said laser light source comprises at least one multimode laser diode.

4. The medical laser apparatus of claim 1, wherein said light pipe comprises a hexagonal shaped fused silica light pipe.

5. The medical laser apparatus of claim 1, wherein said optical diffuser comprises a holographic diffuser.

6. The medical laser apparatus of claim 1, wherein said optical diffuser comprises a micro-lens array.
